(19) United States
(12) Patent Application Publication
(10) Pub. No.: US 2024/0203526 A1
(43) Pub. Date: Jun. 20, 2024

(54) SUBSTRATE SEQUENCE DESIGN WORKFLOW FOR THE RNAI-MEDIATED MULTI-SITE REGULATION OF GENOMIC AND SUB-GENOMIC VIRAL RNAS

(71) Applicant: Speratum Biopharma, Inc., Dover, DE (US)

(72) Inventors: Christian Roberto Marin-Muller, Dover, DE (US); Osvaldo Vega-Martinez, Dover, DE (US); Juan Carlos Valverde-Hernandez, Dover, DE (US)

(73) Assignee: Speratum Biopharma, Inc., Dover, DE (US)

(21) Appl. No.: 18/556,623

(22) PCT Filed: Apr. 19, 2022
(86) PCT No.: PCT/US2022/025440

§ 371 Date: Oct. 20, 2023

Related U.S. Application Data
(60) Provisional application No. 63/177,216, filed on Apr. 20, 2021.

(51) Int. Cl. G16B 30/00; C12N 15/113

(52) U.S. Cl. CPC G16B 30/00; C12N 15/1131; C12N 2310/14

(57) ABSTRACT

A method may include selecting an RNA or a set of RNAs; defining a minimum and a maximum target RNAi transcript hybridization length of the RNA or set of RNAs; and executing a computer algorithm, wherein the computer algorithm determines either a most abundant nucleotide sequences in the transcript or set of transcripts with a length that matches the minimum target RNAi transcript hybridization length.

### CROSS-REFERENCE TO RELATED APPLICATIONS

This application claims priority from U.S. Provisional Patent Application No. 63/177,216, filed on Apr. 20, 2021, the contents of which are incorporated herein by reference.

### FIELD OF THE DISCLOSURE

The present disclosure is in the field of treatments against viruses and diseases. Specifically, treatments utilizing ribonucleic acid (RNA), such as RNA-interference (RNAi) molecules.

### INTRODUCTION

Coronaviruses, and in particular SARS-COV-2, have caused a viral outbreak leading to a worldwide pandemic of Covid-19 illness. Current circumstances globally have resulted in a worldwide public health emergency.

As a human pathogen, according to the World Health Organization (WHO), as of August 2020, over eighteen (18) million cases of Covid-19 have been confirmed globally, with at least 687,000 deaths.

It is now known and understood that SARS-COV-2 is readily transmitted from human to human, spreading to multiple continents and leading to the WHO's declaration of a Public Health Emergency of International Concern (PHEIC) on 30 Jan. 2020.

While efficacy is critical to treating the SARS-COV-2 pandemic, such treatments must also be shown to be safe. Indeed, smaller safety trials must be run to demonstrate safety before engaging in widespread efficacy trials, resulting in additional time.

It would be desirable, therefore, to provide treatments with increased chance of efficacy and safety, in order to maximize the likelihood of success and potential time to approval for treatment of SARS-COV-2.

It would be further desirable to design RNAi molecules that blend siRNA and miRNA characteristics. The inventors have recognized a need to design a workflow that makes a specific multi-match site, extended seed sequence RNAi design possible. In an embodiment of the present disclosure, the designed RNAi will tend to increase silencing specificity while maximizing the number of complementarity sites in the selected target RNA. This combination of properties is especially useful in treating RNA based viruses since these may have high mutation rates and siRNA silencing methods will tend to be fragile. Likewise, mRNA with high mutation rates in cancer may be fragile to siRNA silencing methods but, susceptible to this proposal.

### SUMMARY

In an aspect of the present disclosure a method may include selecting an RNA or a set of RNAs; defining a minimum and a maximum target RNAi transcript hybridization length of the RNA or set of RNAs; and executing a computer algorithm. The computer algorithm may determine either a most abundant nucleotide sequences in the transcript or set of transcripts with a length that matches the minimum target RNAi transcript hybridization length.

In an embodiment, the method may further include selecting an RNAi guide sequence based on an index that represents a probability of the RNAi having silencing capabilities against the RNA or set of RNAs.

In another embodiment, the method may only consider nucleotide sequences with a guanine-cytosine (“GC”) content higher than 35%.

In yet another embodiment, for each of the nucleotide sequences generated, a potential reverse complementary guide strand sequence of the RNAi is generated. The potential reverse complementary guide strand sequence of the RNAi may be generated by producing an average nucleotide sequence with the maximum target RNAi transcript hybridization length from the nucleotide sequences of a maximum length at the sites where a given small nucleotide sequence is present in the transcript or set of transcripts.

In a further embodiment, each of the potential reverse complementary guide strand sequence of the RNAi may be qualified based on certain characteristics. These certain characteristics may include a hit feasibility index sum, or a custom index. The custom index may represent a probability of a given potential hybridization site to have biological significance, or a probability of an RNAi having silencing capabilities against the RNA.

In an aspect of the present disclosure, a system may include a computer algorithm. The computer algorithm may determine either a most abundant nucleotide sequences in a transcript or set of transcripts with a length that matches a minimum target RNAi transcript hybridization length. The minimum target RNAi transcript hybridization length and a maximum target RNAi transcript hybridization length of the RNA or set of RNAs may be defined from a selected RNA or a set of RNAs.

In an embodiment, the computer algorithm may further select an RNAi guide sequence based on an index that represents a probability of the RNAi having silencing capabilities against the RNA or set of RNAs.

In another embodiment, the system may only consider nucleotide sequences with a GC content higher than 35%.

In yet another embodiment, for each of the nucleotide sequences generated, a potential reverse complementary guide strand sequence of the RNAi may be generated.

In a further embodiment, the potential reverse complementary guide strand sequence of the RNAi may be generated by producing an average nucleotide sequence with the maximum target RNAi transcript hybridization length from the nucleotide sequences of a maximum length at the sites where a given small nucleotide sequence is present in the transcript or set of transcripts.

In an embodiment, each of the potential reverse complementary guide strand sequence of the RNAi may be qualified based on certain characteristics. These certain characteristics may include a hit feasibility index, or a custom index. The custom index may represent a probability of a given potential hybridization site to have biological significance, or a probability of an RNAi having silencing capabilities against the RNA.

In another aspect of the present disclosure, a computer-readable storage medium may have data stored therein representing software executable by a computer, the software may have instructions to determine either a most abundant nucleotide sequences in a transcript or set of transcripts with a length that matches a minimum target RNAi transcript hybridization length. The minimum target RNAi transcript hybridization length and a maximum target RNAi transcript hybridization length of the RNA or set of RNAs may be defined from a selected RNA or a set of RNAs.

### BRIEF DESCRIPTION OF THE DRAWINGS

The drawings should, in no way, be construed as limitations on embodiments of the disclosed invention.

FIG. 1 illustrates an exemplary embodiment of RNAi design workflow, according to various embodiments of the present disclosure.

FIG. 2 illustrates an exemplary embodiment of hybridization sites for the proposed RNAi along the Ebola gRNA, according to various embodiments of the present disclosure.

FIG. 3 illustrates an exemplary embodiment of SARS-COV-2 genomic variability effect over the silencing potential of the designed RNAi, according to various embodiments of the present disclosure.

FIG. 4 illustrates an exemplary embodiment of potential hybridization sites for the proposed RNAi along the Ebola gRNA, according to various embodiments of the present disclosure.

FIG. 5 illustrates an exemplary embodiment of potential hybridization sites for the proposed RNAi along the rabies gRNA, according to various embodiments of the present disclosure.

FIG. 6 illustrates an exemplary embodiment of potential hybridization sites for the proposed RNAi along the H1N1 gRNA, according to various embodiments of the present disclosure.

FIG. 7 illustrates an exemplary embodiment of potential hybridization sites for the proposed RNAi along the RSV gRNA, according to various embodiments of the present disclosure.

FIG. 8 illustrates a graph showing a reduction in luciferase activity following transfection of sequence “D.”

FIG. 9 illustrates a graph showing no cytopathic effects through MTS assay at 48 hours post transfection.

FIG. 10 illustrates a graph showing a reduction in protein N mRNA following transfection of sequence “A.”

FIG. 11 illustrates a graph showing a reduction in protein N mRNA following transfection of sequence “S.”

FIG. 12 illustrates a graph showing no significant immune response after exposure to sequence A or D when compared with negative controls (−).

### DETAILED DESCRIPTION

For this disclosure, singular words should be construed to include their plural meaning, unless explicitly stated otherwise. Additionally, the term “including” is not limiting. Further, “or” is equivalent to “and/or,” unless explicitly stated otherwise. Although, ranges may be stated as preferred, unless stated explicitly, there may exist embodiments that operate outside of preferred ranges.

The present disclosure may be further understood with reference to the following description and the appended drawings, wherein like elements are referred to with the same reference numerals. The exemplary embodiments of the present disclosure relate to novel treatments against viruses and diseases, including but not limited to, SARS-COV-2, Ebola, H1N1, RSV, rabies, and any other suitable virus or medical condition. In an embodiment, ribonucleic acid (RNA), such as RNA-interference (RNAi) molecules are utilized. In an embodiment, the RNAi molecules may be used for the downregulation of viral RNA translation.

Further exemplary embodiments provided herein are novel methods for generating proposed RNAi molecules. Further exemplary embodiments provided herein are novel methods for generating potential sequences for RNAi that may be used against many other viruses, diseases and ailments, including but not limited to, Ebola, H1N1, RSV and Rabies.

According to various embodiments, the present disclosure may include a method for generating RNAi sequences with the capacity to target selected RNA by hybridizing at multiple sites. The method may include the steps: selecting an RNA or a set of RNAs; defining what is the minimum and the maximum RNAi—target transcript hybridization length to consider relevant; and executing a computer algorithm. The computer algorithm may proceed to determine which are the most abundant nucleotide sequences in the transcript or set of transcripts with a length that matches the minimum RNAi—transcript hybridization length defined in the previous step. In an embodiment, only the sequences with a GC content higher than 35% are considered. In an embodiment, for each of the small nucleotide sequences generated in the previous step, potential reverse complementary guide strand sequence of the RNAi is generated.

According to various embodiments, potential reverse complementary guide strand sequence of the RNAi may be generated using a variety of options, which may include, but not limited to, any or all of the following options: 1) Generated by producing the average nucleotide sequence with the maximum length defined in the value set in a previous step from the nucleotide sequences of the maximum length at the sites where the given small nucleotide sequence is present in the transcript(s) set in another step; 2) the list of the nucleotide sequences of the maximum length at the sites where the given small nucleotide sequence is present in the transcript(s) set in the previous step; 3) generated by progressively adding nucleotides to the 5′ UTR end of the given small nucleotide sequence until the nucleotide length reaches the maximum length defined in the value set in the earlier step, wherein each nucleotide added contrary to other nucleotide in the basis that the generated sequence is more frequent in the present in the transcript(s) set in this step and/or another step. The nucleotide may be added based on other criteria such as custom indexes that qualify the capacity of a given sequence or its reverse complement to generate hybridizations against a given sequence; 4) any other suitable method that considers the criteria mentioned previously or additional criteria that promotes a high probability of the RNAi hybridizing and silencing the target RNA; and/or 5) any modification or combination of the previously mentioned option or any other criteria that promotes a high probability of two sequences hybridizing.

Further, each of the potential the reverse complementary guide strand sequence of the RNAi may be qualified based on certain characteristics, including but not limited to: 1) The hit feasibility index sum, which may calculate the sum of the hit feasibility index for each potential hybridization site that the guide strand sequence of the RNAi may have based on complementarity; the sites where the guide strand is complementary to the transcript(s) set in this step and/or another step may be identified; the hybridization sites may have a length that spans from the minimum length and the maximum length defined in this step and/or another step, starting in the first nucleotide of the guide sequence; various characteristics are extracted from the start site, such as the AU content near the hybridization site and hybridization expected Gibbs free energy (ΔG) value; and/or the hit feasibility index may be calculated by multiplying the AU content by the negative ΔG value; or 2) any custom index that represents the probability of the given potential hybridization site to have biological significance and ability to resume the capacity of the proposed guide RNAi sequence to silence the RNA defined in this step and/or another step. In an embodiment, if desired, the effect of genetic variability may be given a set of variants from the selected RNA in this step and/or another step. If desired, potential silencing off target effects could be accessed against a specific species.

According to an embodiment, the method of the present disclosure may include selecting the RNAi guide sequence based on the index that represents the probability of the RNAi having silencing capabilities against the RNA. In an embodiment, other selection criteria may also be included in the selection process such as the potential effect of genetic variability on the RNAi hybridization capacities and the off-target effects in each species.

According to an embodiment, the method of the present disclosure may comprise designing and producing RNAi molecules whose seed sequence starts at position 1 of the generated sequences. The RNAi molecules may be or primary, precursor or mature RNAi nature. The processing route may be canonical or non-canonical. It may be used as a mimic or an expression vector. It may be chemically synthesized or biologically produced. The RNAi molecules may contain non-canonical chemical variants such as phosphorothioate, 2′ methylation, 2′ F, and others. In an embodiment, the invention of the present disclosure may utilize software with a graphical user interface or without a graphical user interface. Such software may use any programming language and/or may make use of the algorithm described in this step and/or another step; and/or RNAi that targets SARS-COV-2 gRNA and derived RNA, generated through the workflow mentioned in point 1. In an embodiment, the RNAi passenger sequences may be 40% like the sequences 5′-CACCAUUACUAGGUU-3′, 5′-GUUGAGUCAGAGCUA-3′, 5′-CAUUAGUAGGGUUGA—3′.

Small-interfering RNAs (siRNAs) may be designed to bind to specific target sequences. Off-target effects may occur through non-specific binding, and present a limitation to siRNA design. MicroRNAs (miRNAs) may be naturally occurring RNAi molecules. They may differ from siRNAs in that an miRNA may have evolved to target multiple different targets simultaneously, through virtue of imperfect pairing. This imperfect pairing may lead to differential regulatory responses that differ from the canonical siRNA regulation. In accordance with an embodiment, the RNAi molecules described here may be designed as synthetic molecules in a manner similar to siRNAs, but taking into account the permissibility of imperfect pairing as with an miRNA. Thus, the antiviral oligonucleotides generated provide broader, more flexible targeting, and can anticipate off-target effects.

SARS-COV-2 may be found in the order Nidoviridae, due to its unique, nested replication strategy of 30 kb, or similarly-sized single-stranded viral RNA may be processed into many smaller messenger RNAs (mRNAs), each encoding multiple proteins. The SARS-COV-2 mRNAs may contain a 3′UTR, and the targeting sites may remain intact even once the nested RNA replication process has begun. Thus, in accordance with the disclosure, an RNAi-based attack against viral RNA is proposed, to target such nested sequences.

In accordance with an embodiment of the disclosure, proposed are siRNA and miRNA sequences including one or more of: strand-specific RNA sequences (ssRNA), non-canonical processing; no passenger strands; target minimization in human genome in order to maximize targets in viral genome, administrable with any suitable nucleic acid delivery vehicle; elevated silencing rates, short processing routes to bioactive states, multiple targeting sites to reduce genetic variability between strains, simplified production and isolation/purification in comparison to standard duplexes, and insertable into alternative siRNA construct design.

In other embodiments, through the proposed workflow, canonical, non-canonical, precursor, mature or primary RNA may be designed, since the main output may be the seed sequence containing guide sequence. In an embodiment, once the algorithm outputs the guide sequence, any RNAi architecture may be incorporated.

In accordance with principles of the disclosure, a genomic understanding of SARS-COV-2 may be used to develop drugs and treatments tailored to treating the novel coronavirus associated with Covid-19. Thus, viral RNA of SARS-COV-2 may be targeted with an anti-SARS-COV-2 treatment: siRNA.

The siRNA may be specifically designed with the ability and potential to act on multiple target sites on the RNA of SARS-COV-2. In particular, the siRNA may be designed to act on a plurality of sites on (1) the 3′UTR region of mRNA, and (2) the coding region (CDS) of SARS-COV-2 RNA. Due to its location immediately after the translation termination codon, 3′UTR mRNA may be responsible for regulating gene expression, and are therefore well suited for receiving a therapeutic. By targeting such sites with siRNA, Ago2 mediated-cleavage may occur, which may result in the destruction of viral RNA prior to ribosome entry, thereby resulting in reduction viral replication of SARS-COV-2. Alternatively, inhibition of viral RNA translation may occur, through repression of ribosome drop-off or sequestering of the viral RNA, all through interaction with the target sequences.

RNA viruses may be likely to mutate, and the use of RNAi-inducing molecules against such viruses may result in imperfect matches, due to the specific complementarity needed for siRNA activity. Moreover, siRNAs tend to require integrity of a relatively long sequence, which makes mutations even more cumbersome, in order to appropriately match with an intended target RNA.

Accordingly, the present disclosure contemplates addressing likely mutations and requirements for high complementarity of siRNA through the generation of a synthetic oligonucleotide that behaves like an miRNA. miRNA may be less specific, and more suitably, may match with a greater number of match sites per target RNA. As such, due to increased promiscuity for regulatory target sites, miRNA regulation to specific RNA targets may be better suited for genetic variability and mutation, as compared to siRNA targeting. Due to the presence of only one match site, siRNA complementary site deletion may eliminate the siRNA's silencing effect on the target RNA, increasing the likelihood of mutation destroying its regulation. In contrast, miRNAs, with multiple complementary sites on target, are capable of maintaining regulation even when one match site is deleted or changed. In one embodiment, however, a single target site with the right sequence may be sufficient for effective regulation.

Therefore, in one embodiment, a proprietary RNAi may be provided, specifically formulated to increase silencing specificity while maximizing the number of complementary sites in the selected RNA target. As such, RNA-based viruses may be able to be treated effectively, even with their high mutation rates. Accordingly, in accordance with numerous embodiments, the disclosure may provide systems and methods identifying potential targeting sites, and may provide systems and methods for designing target sequences, in order to maximize antiviral targeting, while decreasing the risk of mutation and target destruction. In some embodiments, combinations of RNAi molecules may be used. Either miRNA or siRNA-designed molecules may be used alone. The RNAi molecules may target any or multiple portions of the viral genome.

In an embodiment of the present disclosure, synthetic RNAi molecules were designed for SARS-COV-2 RNA silencing using bioinformatics analysis and prediction tools and the No Pass Mimic RNAi administration platform. In an embodiment of the present disclosure, at least two exemplary RNAi design workflows were implemented, one may involve searching for sequences that putatively will silence the virus in a microRNA using a Python algorithm while another may involve the design of siRNAs directed towards the regulation of the virus through the 3′UTR.

TABLE 1
RNAi properties and their association with typical siRNAs and miRNAs.

| Property | siRNA | miRNA |
|---|---|---|
| Specificity (off-target effects) - | Longer match site stabilizes the | Shorter match sites make the |
| length of complementarity | RNAi-RNA interaction, which | RNAi-RNA interaction less |
| match: stability | reduces possible off target | stable when compared to siRNA |
| | effects | |
| Specificity (off-target effects) - | Longer specific nucleotide | Since the silencing in miRNA is |
| length of complementarity | sequences have a smaller | based on the seed sequence, and |
| match: match length | occurrence than short sequences | the seed sequence is 7nts long, |
| | (based on probabilities). | it's more likely that off targets |
| | Therefore, off targets will tend | have similar match length when |
| | to be attributed to partial | compared to desired molecule |
| | matches. | silencing. |
| Specificity (off-target effects) - | Seed sequence complementarity | Seed sequence complementarity |
| seed sequence complementarity | is avoided by reducing the | is avoided by reducing the |
| stability | stability of the RNAi-RNA | stability of the RNAi-RNA |
| | interaction | interaction |
| RNA targeting | Is designed to target single RNA | Is usually of natural occurrence |
| | molecules | and its targeting is promiscuous |
| Amount of complementarity | One | May have many, only in the |
| sites inside a transcript | | 3′UTR region, 3 match sites are |
| | | common |

Table 1, above, represents the selected sequences, according to an embodiment of the present disclosure.

For the first strategy, in an embodiment, a Python 3.7 script may be used to obtain a list of potential RNAi sequences based on a series of features. These synthetic RNAi molecules may be intended to exploit RISC silencing by seed sequence pairing and beyond seed sequence pairing to the transcripts of interest, these being the virus' genomic RNA and sub-genomic RNA. This may involve the design of a guide strand based on the criteria mentioned previously, in hand with common rules for RNAi design.

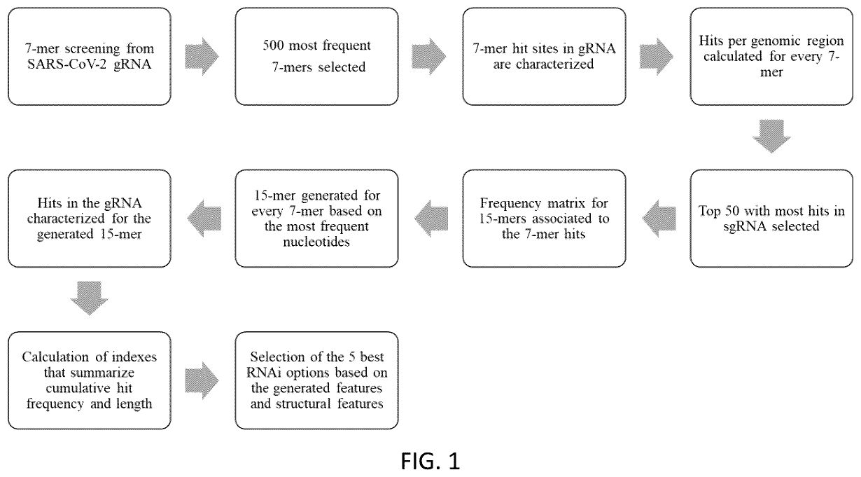

According to an exemplary embodiment of the present disclosure, the workflow implemented for the identification of the potential RNAi sequences may be reviewed in FIG. 1. In an embodiment, the potential 7 nucleotides-long seed sequences may be identified by searching screening for the most 7-mers in the SARS-COV-2 genome using the Python 3.7 script. In such an embodiment, the RefSeq genome with the accession number NC_045512.2 may be used. The search may include 5′UTR and CDSs, since studies have found that the RISC complex may, in some cases, silence transcripts through regulation in the previously mentioned regions (Hausser et al., 2013; Lytle et al., 2007). In an embodiment, the 500 most frequent 7-mers were selected for further analysis.

FIG. 1 illustrates an exemplary embodiment of RNAi design workflow, according to various embodiments of the present disclosure. At a first step, identification of potential RNAi sequences may be performed. Thus, a potential 7-nucleotide seed sequence may be searched for, by searching the most 7-mers in the target RNA. For example, 5′UTR and CDS may be evaluated, due to the RNA-induced silencing process causing a silencing of transcripts through regulation in 5′UTR and CDS. Details of this may be found in Hausser, J., Syed, A. P., Bilen, B., & Zavolan, M. (2013), “Analysis of CDS-located miRNA target sites suggests that they can effectively inhibit translation,” Genome Research, 23(4), 604-615, https://doi.org/10.1101/gr.139758.112, and Lytle, J. R., Yario, T. A., & Steitz, J. A. (2007), “Target mRNAs are repressed as efficiently by microRNA-binding sites in the 5′ UTR as in the 3′ UTR,” Proceedings of the National Academy of Sciences of the United States of America, 104(23), 9667-9672, https://doi.org/10.1073/pnas.0703820104, the contents of which are incorporated by reference herein in their entirety. After compiling the 7′mers, the 500 most frequent ones may be selected for further analysis. In an alternate exemplary embodiment, for the other viruses, including but not limited to Ebola, a potential 6-mers may be searched for in the target RNA instead of 7-mers, as discussed in further detail in subsequent paragraphs of this disclosure.

In a second step of an embodiment, every position in the genomic RNA sequence where the selected 7-mers are present, known as the hit site, may be registered alongside with the position, the name of the genomic region where the 7-mer was found and the 15-mer starting at the 7-mer start position were registered. Fifty 7-mers with the most hits regions may be selected.

In a third step of an embodiment, the amount of hit sites per genomic region may be calculated for every 7-mer. The 50 7-mers with most hits in regions other than the ORF1 may be selected. This filter may be generated to select the 7-mers which may most likely show silencing in the in vitro models that don't involve viral infection to cultured cells.

In a fourth step of an embodiment, using the registered 15-mers, which do not come from ORF1 hits, a nucleotide position frequency matrix may be generated, and a 15-mer may be generated. Each 7-mer may be based on the most frequent nucleotide for each position, with the sequence corresponding to the proposed sense strand for the RNAi. Accordingly, non-canonical base paring and beyond seed sequence pairing may be promoted.

Although 15-mers may be used in one exemplary embodiment, the present disclosure is not limited to this length. It should be understood that the standard length for siRNA is from 18 to 20 nucleotides of the targeting sequence, and the described method could generate sequences with these characteristics.

In a fifth step of an embodiment, hits of generated 15-mer sequences may be predicted for the presumed RNAi molecule based on perfect matching. Thus, perfect seed sequence and beyond seed sequence matches may be searched. For examples, matches of 5, 6, 7, 8, 9, 10, 11, 12, 14 and 15 nucleotides long may be registered. Although a large body of research may support and validate the effect of 6, 7 and 8 nucleotide long matches, shorter RNAi matches with transcripts such as 5 nucleotide long match and larger matches such as 10 and 15 matches may have been shown to be involved in the silencing of RNA molecules (Broughton, J. P., Lovci, M. T., Huang, J. L., Yeo, G. W., & Pasquinelli, A. E. (2016), “Pairing beyond the Seed Supports MicroRNA Targeting Specificity,” Molecular Cell, 64(2), 320-333, https://doi.org/10.1016/j.molcel.2016.09.004; Chandradoss, S. D., Schirle, N. T., Szczepaniak, M., Macrae, I. J., & Joo, C. (2015), “A Dynamic Search Process Underlies MicroRNA Targeting. Cell, 162(1), 96-107,” https://doi.org/10.1016/j.cell.2015.06.032; Chipman, L. B., & Pasquinelli, A. E. (2019), “miRNA Targeting: Growing beyond the Seed,” Trends in Genetics, 35(3), 215-222, https://doi.org/10.1016/j.tig.2018.12.005, the contents of all are incorporated by reference in their entireties).

In a sixth step of an embodiment, for every registered hit site, a hit feasibility index may be calculated. A weight may be assigned to every hit, based on its match sequence ΔG value and the AU content proportion 30 nt upstream and downstream from the hit site.

Thus, in embodiments where the structure of the seed sequence is highly responsible for its target silencing capacities, as lower ΔG seed sequence pairing values tend to correlate with higher suppression levels (Wang, X. (2014), “Composition of seed sequence is a major determinant of microRNA targeting patterns.” Bioinformatics, 30(10), 1377-1383, https://doi.org/10.1093/bioinformatics/btu045, incorporated herein by reference in its entirety.

Where higher AU contents are found near the RNAi binding sites, this may correlate with more transcript and protein silencing, alluding to usually a less stable secondary structure (Bartel, D. P. (2009), “MicroRNAs: Target Recognition and Regulatory Functions,” Cell, 136(2), 215-233, https://doi.org/10.1016/j.cell.2009.01.002; Laganà, A., Acunzo, M., Romano, G., Pulvirenti, A., Veneziano, D., Cascione, L., . . . Croce, C. M. (2014), “MiR-Synth: A computational resource for the design of multi-site multi-target synthetic miRNAs,” Nucleic Acids Research, 42(9), 5416-5425, https://doi.org/10.1093/nar/gku202; Peterson, S. M., Thompson, J. A., Ufkin, M. L., Sathyanarayana, P., Liaw, L., & Congdon, C. B. (2014), “Common features of microRNA target prediction tools,” Frontiers in Genetics, 5(FEB), 1-10, https://doi.org/10.3389/fgene.2014.00023; Yan, Y., Acevedo, M., Mignacca, L., Desjardins, P., Scott, N., Imane, R., . . . Major, F. (2018), “The sequence features that define efficient and specific hAGO2-dependent miRNA silencing guides,” Nucleic Acids Research, 46(16), 8181-8196, https://doi.org/10.1093/nar/gky546. The contents of all of these references are incorporated herein by reference in their entireties).

$${H}_{{n}_{m}} = ( {p ( A U )}_{{n}_{m}} \times - Δ {G}_{{n}_{m}} )$$

Disclosed above is an exemplary equation where n is the generated 15-mer used to predict the m hit site, H is the hit feasibility, ΔG is the free energy required for the match to happen considering only base pairing and is the p(AU) proportion 30 nt upstream and downstream from the hit site. Next, two indexes may be calculated for the generated 15-mers: IGₘ=Σₖ₌₁¹⁵(Σ_{M=1}^{A}Hₙ_{ₘ}), IG may be the general index which summarizes the effect of the hits, A is the amount of hits for the n generated 15-mer of the match, and is the hit amount per region, where r represents the region.

On the other hand, IIVₘ=Σₖ₌₁¹⁵(Σₘ₌₁^{B}Hₙ_{ₘ}), is an exemplary equation where IIV is the in vitro index. It summarizes the same factors that it considers, with the exception that B only includes the hits inside the virus' 3′UTR, 5′UTR, N gene, and S gene. It may be generated to guide the selection of the RNAi sequences considering the in vitro models previously mentioned limitations.

In the seventh step of an embodiment, a matrix may be developed featuring: the 7-mer, it's generated 15-mer or proposed antisense strand, the sense or guide strand, the RNAi proposed sequence, the seed sequence GC content, the guide strand GC content, as well as all aforementioned indexes, and the hits in the 3′UTR, 5′UTR, N gene, and S gene. Three sequences may be selected based on the general balance of the features described previously and other recommended features for the design of RNAi molecules. Human transcripts that may be silenced by seed off-target effects are searched using the Custom microRNA Prediction functionality of the miRDB platform (Chen & Wang, 2020).

TABLE 2

| RNAi Identifier | Sequence |
|---|---|
| Anti-SC2-A | UCACCAUUACUAGGUUUG |
| Anti-SC2-B | UGUUGAGUCAGAGCUAUG |
| Anti-SC2-C | UCAUUAGUAGGGUUGA |
| Anti-SC2-D | UUCACUGUACACUCGA |
| Anti-SC2-E | UUUACACAUUAGGGCU |

Illustrated above in Table 2 are five RNAi SARS-COV-2 silencing sequences, in accordance with an exemplary embodiment. These sequences may incorporate the NPM architecture. However, in another embodiment, RNAi that targets SARS-COV-2 gRNA and derived RNA, may be generated through the workflow mentioned. In an embodiment, the RNAi guide sequences may be 40% like the sequences A-5′-CACCAUUACUAGGUU-3′, B-5′-GUUGAGUCAGAGCUA-3′, C-5′-CAUUAGUAGGGUUGA—3. RNAi that targets SARS-CoV-2 gRNA and derived RNA, may be generated through a workflow different than the one mentioned previously (this sequence may be generated through the standard process of generating siRNA). In an embodiment, the RNAi guide sequences may be 40% like the sequence D-5′UCACUGUACACUCGA-3.

TABLE 3

| RNAi Identifier | On 3′UTR | On CDS |
|---|---|---|
| Anti-SC2-A | 0 | 17 |
| Anti-SC2-B | 1 | 23 |
| Anti-SC2-C | 1 | 8 |
| Anti-SC2-D | 1 | 0 |
| Anti-SC2-E | 1 | 0 |

Illustrated above in Table 3 are the number of matching sites of RNAi SARS-CoV-2 silencing sequences, according to an exemplary embodiment of the present disclosure.

TABLE 4

| | | | GC | |
|---|---|---|---|---|
| | | | content | GC |
| | Guide | Passenger | of seed | content |
| Identifier | sequence | sequence | sequence | of guide |
| Negative | CACAACCA | UUCUUGGUG | 57.14 | 47 |
| control* | CCAAGAA | GUUGUG | | |
| NPM-SC2-A | CACCAUUA | AACCUAGUA | 43 | 40 |
| | CUAGGUU | AUGGUG | | |
| NPM-SC2-B | GUUGAGUC | UAGCUCUGA | 43 | 47 |
| | AGAGCUA | CUCAAC | | |
| NPM-SC2-C | CAUUAGUA | UCAACCCUA | 29 | 40 |
| | GGGUUGA | CUAAUG | | |

*This sequence wasn't generated through the proposed workflow. Its design is instead based on cel-mir-67-3p.

Illustrated above in Table 4 are the general properties of control RNAi generated through the verification that they will not interact with the viral mRNA through the proposed workflow, according to an exemplary embodiment of the present disclosure.

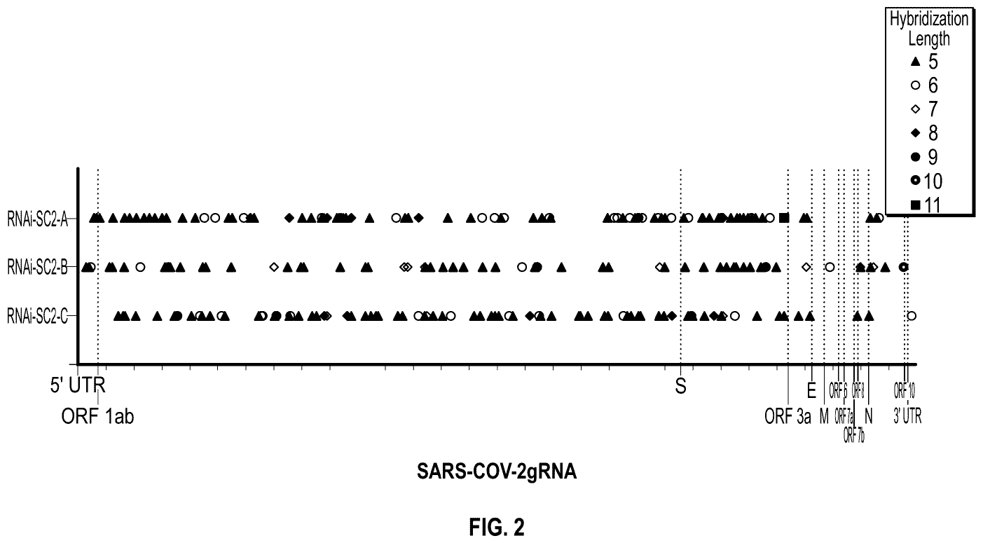

FIG. 2, illustrates hybridization sites for the proposed RNAi along the Ebola gRNA according to an exemplary embodiment of the present disclosure. The minor tick marks may represent 1 kilobase.

The viral genetic variability may have an effect on the designed RNAi silencing capacities. According to an exemplary embodiment of the present disclosure, whole genomes of SARS-COV-2 may be extracted from the NCBI Virus Sequences for discovery platform with the following query:
Collection date: from 13-Apr. -2020 to 21-Jan. -2021
Release date: from 13-Apr. -2020 to 21-Jan. -2021
Host: Humans (taxid: 9605)
Nucleotide completeness: complete
Sequence type: GenBank
Sequence length: minimum 29000
Virus: SARS-COV-2 (taxid: 2697049)

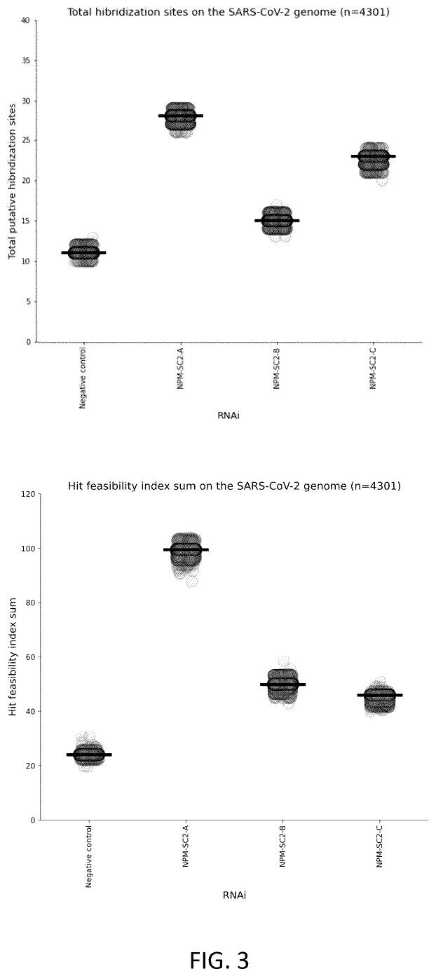

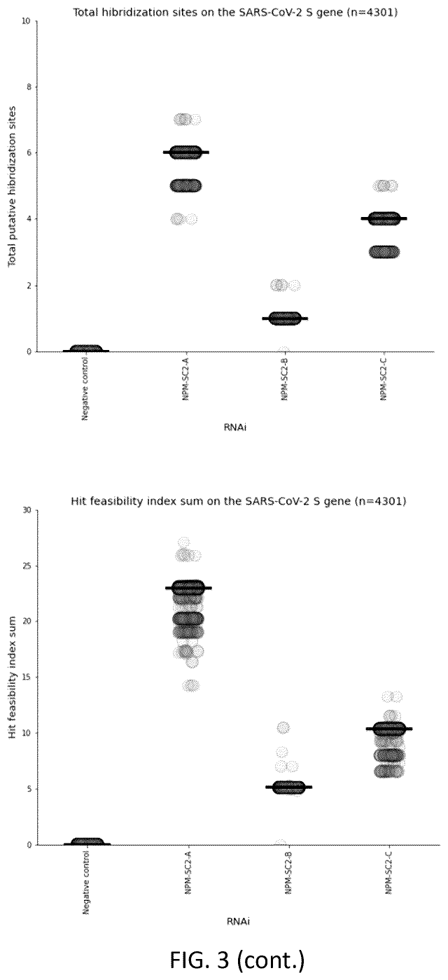

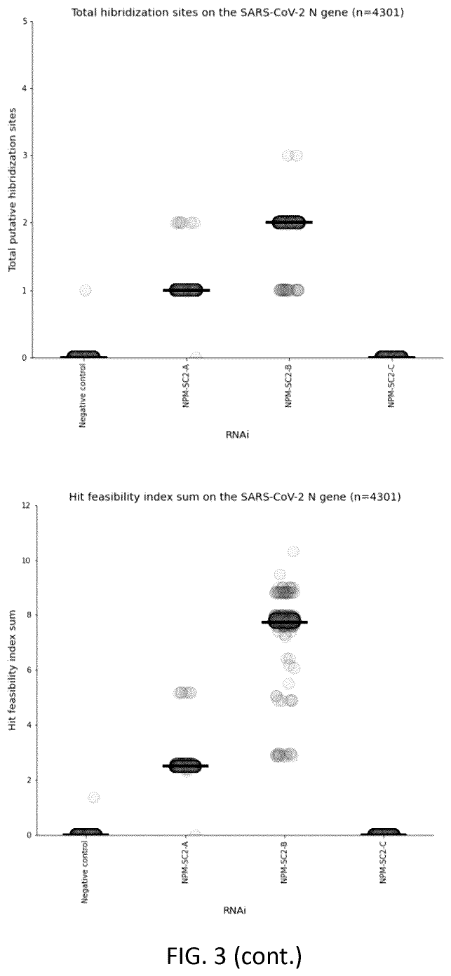

According to this embodiment, a total of 4301 genomes may be extracted. The putative hybridization sites of 6 to 15 nucleotides long may be identified for every genome. The total hit amount and the total hit feasibility index may be determined (the index as indicated in the following section). This may be executed for the 3 designed RNAi reported previously and one negative control named NPM-NC-Cel. FIG. 3 summarizes the obtained results, according to an exemplary embodiment.

FIG. 3 illustrates SARS-COV-2 genomic variability effect over the silencing potential of the designed RNAi, according to an exemplary embodiment of the present disclosure. The reference genome values are represented with the dash marker. Variant genomes are represented with the circle markers.

According to another exemplary embodiment, the SARS-COV-2 workflow may be modified as follows: The workflow implemented for the identification of the potential RNAi sequences may be reviewed in FIG. 1. According to an embodiment, a potential 6-mers may be searched for in the target RNA instead of 7-mers. According to such an embodiment, first, the potential 6 nucleotides-long seed sequences may be identified by searching screening for the most 6-mers in the target RNA. The search included 5′UTR and CDSs since studies have found that the RISC complex may in some cases silence transcripts through regulation in the previously mentioned regions (Hausser, Syed, Bilen, & Zavolan, 2013; Lytle, Yario, & Steitz, 2007). The 500 most frequent 6-mers with a GC content higher than 40% may be selected for further analysis.

Second, according to an embodiment, every position in the genomic RNA sequence where the selected 6-mers are present (hit site) may be registered alongside with the position, the name of the genomic region where the 6-mer was found, and the 15-mer starting at the 6-mer start position. The 75 6-mers with most hits sites may then be selected.

Third, according to an embodiment, using the registered 15-mers, a nucleotide position frequency matrix may be generated and a 15-mer may be generated for each 6-mer based on the most frequent nucleotide for each position. This sequence, alongside the registered 15-mers, may be subsequently tested.

In the next steps of this embodiment, the total amount of possible hybridization sites may be determined. In an embodiment, for each possible hybridization site (or hit site), a hit feasibility index may be calculated in order to rank these sequences by their potential RNAi activity against the selected gRNA and its derived RNA. This strategy may be done to promote non-canonical base pairing and beyond seed sequence pairing.

Fourth, according to an embodiment, using the generated 15-mer sequences, hits may be predicted for the putative RNAi molecule based on perfect matching. Perfect seed sequence and beyond seed sequence matches may be searched, specifically matches from 6 to 22 nucleotides long may be registered. Although a large body of research may support and validate the effect of 6, 7 and 8 nucleotide long matches (Bartel, 2009), both shorter RNAi matches with transcripts such as 6 nucleotide long match and larger matches such as 10 and 15 matches may have been shown to be involved in the silencing of RNA molecules (Broughton, Lovci, Huang, Yeo, & Pasquinelli, 2016; Chandradoss, Schirle, Szczepaniak, Macrae, & Joo, 2015; Chipman & Pasquinelli, 2019; Hart et al., 2018).

Fifth, according to an embodiment, for every registered hit site, a value we name hit feasibility index may be calculated. It may assign a weight to every hit based on its match sequence ΔG value and the AU content proportion 30 nt upstream and downstream from the hit site. Reports may have indicated that the structure of the seed sequence is highly responsible for its target silencing capacities, as lower ΔG seed sequence pairing values may tend to correlate with higher suppression levels (Wang, 2014). Likewise, higher AU contents near the RNAi binding sites may tend to correlate with more transcript and protein silencing, alluding to usually a less stable secondary structure (Bartel, 2009; Laganà et al., 2014; Navarro et al., 2015; Peterson et al., 2014; Yan et al., 2018).

$${H}_{{n}_{m}} = ( {p ( A U )}_{{n}_{m}} \times - Δ {G}_{{n}_{m}} )$$

Disclosed above is an exemplary equation, according to an embodiment, where n is the generated 15-mer used to predict the m hit site, H is the hit feasibility, ΔG is the free energy required for the match to happen considering only base pairing and is the p(AU) proportion 30 nt upstream and downstream from the hit site. In cases where the ΔG was positive, ΔG was converted to 0. Next, two indexes may be calculated for the generated 15-mers, IGₘ=Eₖ₌₁¹⁵(Eₘ₌₁^{A}Hₙ_{ₘ}), where IG is the general index which summarizes the effect of the hits, A is the number of hits for the n generated 15-mer of the match.

Sixth, according to an embodiment, a matrix may be developed that summarizes the following information and features: the 6-mer, it's generated 15-mer or proposed antisense strand, the sense or guide strand, the RNAi proposed sequence, the seed sequence GC content, the guide strand GC content, and the previously mentioned indexes. Human transcripts could potentially be silenced by seed off-target effects searched using the Custom microRNA Prediction functionality of the miRDB platform (Liu & Wang, 2019).

In an embodiment, the proposed workflow may be used in an Ebola approach as follows. According to an embodiment, the Ebola approach may utilize potential RNAi sequences against Ebola and may be generated using the previously mentioned workflow. In an embodiment, the gRNA used may correspond to the sequence registered with the NCBI accession code NC_002549.1. In such an embodiment, the genomic annotation (or position of genes) may be extracted from the annotations linked to the accession code. Table 5, below, illustrates five of the top ranked proposed RNAi according to the hit feasibility index, according to an exemplary embodiment.

TABLE 5
General properties of the anti-Ebola RNAi
generated through the proposed workflow.

| | | | GC | GC | | |
|---|---|---|---|---|---|---|
| | | | content | content | Hit | Amount |
| | Guide | Passenger | of seed | of | feasibility | of |
| Identifier | sequence | sequence | sequence | guide | index | hits |
| RNAi-E01 | CCUCUUGG | UUAGGCCCC | 50 | 60 | 61.4 | 14 |
| | GGCCUAA | AAGAGG | | | | |
| RNAi-E02 | GUUGUCUC | GUGAGUGGA | 50 | 53 | 57.1 | 15 |
| | CACUCAC | GACAAC | | | | |
| RNAi-E03 | UGUCUCCU | CCGGAAGAG | 50 | 60 | 56.7 | 15 |
| | CUUCCGG | GAGACA | | | | |
| RNAi-E04 | UUCCUCAG | GGACAGACU | 50 | 53 | 55.0 | 12 |
| | UCUGUCC | GAGGAA | | | | |
| RNAi-E05 | UGCUGUUG | GGUCGGGCA | 50 | 67 | 54.9 | 14 |
| | CCCGACC | ACAGCA | | | | |

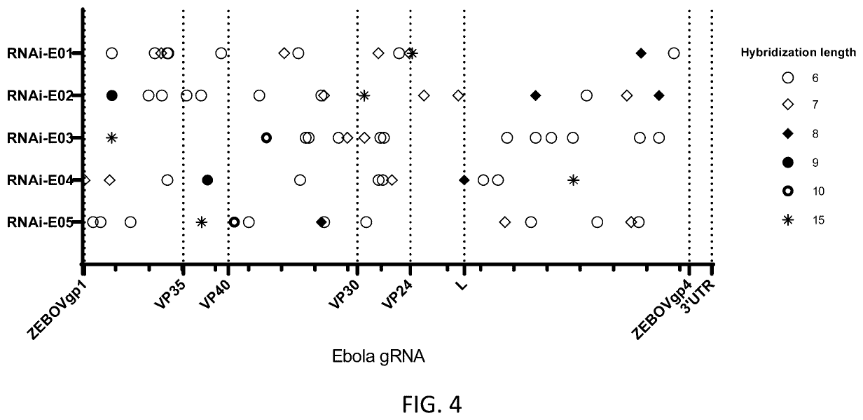

FIG. 4, illustrates potential hybridization sites for the proposed RNAi along the Ebola gRNA, according to an exemplary embodiment. The minor tick marks represent 1 kilobase.

In another embodiment, the proposed workflow may be used in a Rabies approach as follows. According to an embodiment, the Rabies approach utilizes potential RNAi sequences against rabies and may be generated using the previously mentioned workflow. In an embodiment, the gRNA used may correspond to the sequence registered with the NCBI accession code HQ450386.1. In this embodiment, the genomic annotation (position of genes) may be extracted from the annotations linked to the accession code. Table 6, below, illustrates five of the top ranked proposed RNAi according to the hit feasibility index, according to an exemplary embodiment.

TABLE 6
General properties of the anti-rabies RNAi generated
through the proposed workflow.

| | | | GC | GC | | |
|---|---|---|---|---|---|---|
| | | | content | content | Hit | Amount |
| | Guide | Passenger | of seed | of | feasibility | of |
| Identifier | sequence | sequence | sequence | guide | index | hits |
| RNAi-R01 | CCCUCUGG | AAUCCCCCC | 67 | 67 | 65.8 | 13 |
| | GGGGAUU | AGAGGG | | | | |
| RNAi-R02 | CCCCUCUU | GGCUCGCAA | 83 | 73 | 57.0 | 9 |
| | GCGAGCC | GAGGGG | | | | |
| RNAi-R03 | UCUCCUCC | UACCUCUGG | 50 | 53 | 52.8 | 12 |
| | AGAGGUA | AGGAGA | | | | |
| RNAi-R04 | CUUCCCCC | CCCAACUGG | 67 | 67 | 52.3 | 10 |
| | AGUUGGG | GGGAAG | | | | |
| RNAi-R05 | UCCUUCCC | GGAUACCGG | 50 | 60 | 52.0 | 13 |
| | GGUAUCC | GAAGGA | | | | |

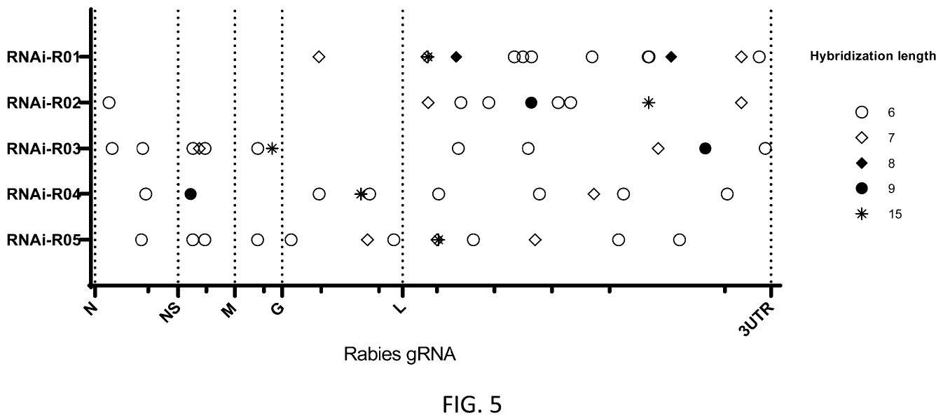

FIG. 5, illustrates potential hybridization sites for the proposed RNAi along the rabies gRNA, according to an exemplary embodiment. The minor tick marks represent 1 kilobase.

In another embodiment, the proposed workflow may be used in an Influenza A virus subtype H1N1 approach as follows. According to an embodiment, the Influenza A virus subtype H1N1 approach may utilize potential RNAi sequences against Influenza A virus subtype H1N1 and may be generated using the previously mentioned workflow. In an embodiment, the 8 gRNA fragments used may correspond to the sequence registered with the NCBI accession codes NC_026436.1 NC_026431.1 NC_026432.1 NC_026433.1 NC_026437.1 NC 026434.1 NC 026435.1 NC_026438.1. In such an embodiment, the sequences may be concatenated in order based on the fragment designated number. Table 7, below, illustrates five of the top ranked proposed RNAi according to the hit feasibility index, according to an exemplary embodiment.

TABLE 7
General properties of the anti-H1N1 RNAi generated
through the proposed workflow.

| | | | GC | GC | | |
|---|---|---|---|---|---|---|
| | | | content | content | Hit | Amount |
| | Guide | Passenger | of seed | of | feasibility | of |
| Identifier | sequence | sequence | sequence | guide | index | hits |
| RNAi-HN01 | UUCCCUCC | ACGAGGGGG | 50 | 67 | 60.4 | 13 |
| | CCCUCGU | AGGGAA | | | | |
| RNAi-HN02 | UGCCUUGA | UGGCGUCUC | 50 | 60 | 60.2 | 14 |
| | GACGCCA | AAGGCA | | | | |
| RNAi-HN03 | UUCCUCUU | GAUUCCCAA | 50 | 47 | 59.4 | 15 |
| | GGGAAUC | GAGGAA | | | | |
| RNAi-HN04 | UUGCCCCC | UCUCCCUGG | 67 | 67 | 57.3 | 11 |
| | AGGGAGA | GGGCAA | | | | |
| RNAi-HN05 | GGCCAUUG | GGGAGAUCA | 67 | 60 | 56.4 | 11 |
| | AUCUCCC | AUGGCC | | | | |

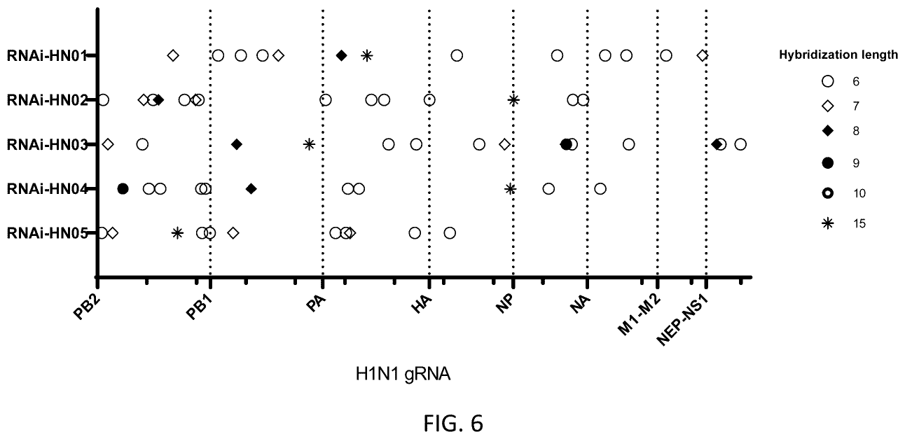

FIG. 6, illustrates potential hybridization sites for the proposed RNAi along the H1N1 gRNA, according to an exemplary embodiment. The minor tick marks represent 1 kilobase.

In another embodiment, the proposed workflow may be used in a Human respiratory syncytial virus A approach as follows. According to an embodiment, the Human respiratory syncytial virus A approach may utilize potential RNAi sequences against Human respiratory syncytial virus A (RSV) and may be generated using the previously mentioned workflow. In an embodiment, the gRNA used may corresponds to the sequence registered with the NCBI accession code MW020599.1. In such an embodiment, the genomic annotation (position of genes) may be extracted from the annotations linked to the accession code. Table 8, below, illustrates five of the top ranked proposed RNAi according to the hit feasibility index, according to an exemplary embodiment.

TABLE 8
General properties of the anti-RSV RNAi generated
through the proposed workflow.

| | | | GC | GC | | |
|---|---|---|---|---|---|---|
| | | | content | content | Hit | Amount |
| | Guide | Passenger | of seed | of | feasibility | of |
| Identifier | sequence | sequence | sequence | guide | index | hits |
| RNAi-RSV01 | UUUGCCCC | GAAAGAUGG | 50 | 47 | 102.2 | 14 |
| | AUCUUUC | GGCAAA | | | | |
| RNAi-RSV02 | UUGCCCCA | UGAAAGAUG | 67 | 47 | 88.2 | 11 |
| | UCUUUCA | GGGCAA | | | | |
| RNAi-RSV03 | UGCCCCAU | AUGAAAGAU | 83 | 47 | 77.6 | 9 |
| | CUUUCAU | GGGGCA | | | | |
| RNAi-RSV04 | GCCCCAUC | GAUGAAAGA | 83 | 53 | 76.7 | 9 |
| | UUUCAUC | UGGGGC | | | | |
| RNAi-RSV05 | UUGUGGUG | GCAAACCCA | 50 | 53 | 66.0 | 18 |
| | GGUUUGC | CCACAA | | | | |

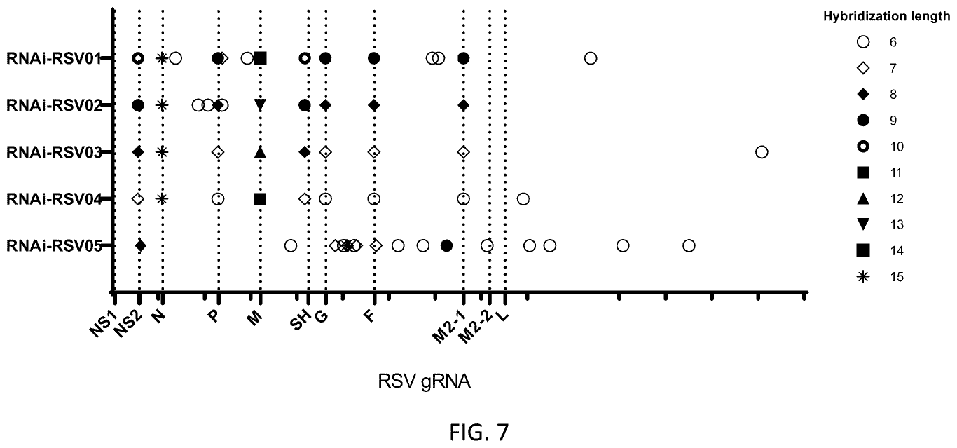

FIG. 7, illustrates potential hybridization sites for the proposed RNAi along the RSV gRNA, according to an exemplary embodiment. The minor tick marks represent 1 kilobase.

As disclosed herein, the disclosure may be modified by chemical modifications, as contemplated. Alternatively, molecular presentation may be modified to administer treatment as an RNAi mimic or as an expression vector, such as a virus, plasmid, or others.

The various embodiments may be modified in combination with various delivery methods, such as LNP, polymeric nanoparticles, aptamer associated delivery, antibody delivery, affimer associated delivery or metal nanoparticles.

Further, treatment may be administered either as linear or circular RNA, or as part of a longer non-coding RNA. Treatment may also be administered as a DNA counterpart, including via a plasmid or other system vector, or shRNA vector system.

The embodiments disclosed herein may combine the benefits of siRNA and miRNA to add capacity to have multiple complementarity sites, as in miRNA, while promoting a high specificity similar to siRNA. Additionally, this may address issues of mutation, and may be used to design RNAi molecules to target either single or multiple selected RNA.

Therefore, the various embodiments may be designed to silence coding and non-coding RNA, or silence single-selected RNA, or combinations of RNA. Further, regions inside the RNA to be targeted may be enriched for target sites. These embodiments may be implemented clinically or non-clinically, and administered alone or in combination with other compounds.

As stated in earlier paragraphs, in an embodiment of the present disclosure, the Python algorithm script may be used in the exemplary workflows. According to an embodiment, the first step is to import libraries. The libraries required include, but are not limited to, the seqlogo: https://pypi.org/project/seqlogo/, numpy, pandas, datetime, os, oligo_melting: https://pypi.org/project/oligo-melting/, random, searborn, and string.

According to this embodiment, the second step may be to define functions. In this embodiment, this step may include, but is not limited to the following operations: (i) fastaToDict: Read fasta-format file, return dict of form scaffold:sequence. Note: Uses only the unique identifier of each sequence, rather than the entire header, for dict keys. —https://gist.github.com/jacob-ogre/5318981; (ii) rnaReverseComplementary: Generates the reverse complementary sequence given RNA from a sequence; (iii) rnaReverseComplementary: Generates the reverse complementary sequence from a given RNA sequence; (iv) locateKmers: Identifies the sites inside the transcript where the kmer is found. The sequence that flanks the kmer position may be recorded. This sequence may eventually allow the maxization of the RNAi match site length. (v) synSeedPassengers: A series of kmers will be filtered out between the execution of the “locateKmers” command and the current command. This algorithm may compile all of the flanking sequence obtained using the defined command “locateKmers” per k-mer. (vi) passengerNucleotideProportion: To maximize the percentage of the RNAi sequence that is complementary to the seed sequence or k-mer match-site against the target RNA, a rest of the RNAi sequence may need to be selected based on the sequences that go after the k-mer sequence. Taking this into consideration, an “average” sequence may be generated for the nucleotides beyond the seed sequence based on the nucleotide frequency in each position after the seed sequence (a nucleotide position probability matrix or PPM may be created to address this necessity). This may be done in 2 different phases (the current function, “passenger NucleotideProportion”, and the following “averagePassenger” function). In the current function a nucleotide position proportion matrix may be generated. (vii) averagePassenger: In order to maximize the percentage of the RNAi sequence that is complementary to the seed sequence or kmer match-site against the target RNA, a rest of the RNAi sequence may need to be selected based on the sequences that go after the k-mer sequence. Taking this into consideration, an “average” sequence may be generated for the nucleotides beyond the seed sequence based on the nucleotide frequency in each position after the seed sequence. This may be done in 2 different phases (“passengerNucleotideProportion” and the current function, “averagePassenger”). (vii) putativeHybridizations: It registers putative matches for every RNAi sequence or average_passenger against the target RNA. The length of the match may be determined. The flanking match-site sequence in the target RNA may be analyzed. The upstream and downstream sequence's AU % may be quantified based on the 30 nts upstream and downstream. Likewise, the dG value may be calculated for each putative uninterrupted match. The likelihood of a single predicted match to have biological relevance may be estimated using the calculated index. (ix) putativeHybCleanUp: The output of the function “putativeHybridizations” is formatted assuring that there aren't more than one entry per match registered. (x) genomeTestSubset: This function may be used in a given case that only a part of the variant sequences are wanted to be analyzed in the light of the potential silencing effect that a set of RNAi could have. It may randomly select a number of genomes defined by the subset_size input variable from the variant genomes dictionary if the subset_size is smaller than the amount of sequences stored in variant_genomes.

While this disclosure has been described in conjunction with the embodiments outlined above, many alternatives, modifications and variations will be apparent to those skilled in the art upon reading the foregoing disclosure. Accordingly, the embodiments of the disclosure, as set forth above, are intended to be illustrative, not limiting. Various changes may be made without departing from the spirit and scope of the disclosure.

### Experimental Data

### SARS-COV-2 In Vitro Surrogate Expression Methods

### Cell Culture

STR validated HeLa cervical cancer cell line was maintained in DMEM medium (D6429, Sigma-Aldrich) supplemented with 10% fetal bovine serum (F2442, Sigma-Aldrich), 1% antibiotic-antimycotic solution (A5955, Sigma-Aldrich), 1% Penincilin Streptomicyn (15140-122, Gibco) and 5 ug/mL Plasmocin™ Prophylactic (ant-mpt, Invivo Gen).

### Plasmid Construct Design

For a first approach of the therapeutic potential of the anti SARS-COV-2 designed miRNA mimics, a Firefly-Renilla luciferases reporter vector and a S-protein/N-protein expression vector may be designed as follows:
(a) For the Firefly-Renilla luciferases reporter vector, the SARS-COV-2 universal 3′UTR sequence will be synthetized and cloned in the 3′ end of the Firefly luciferase transcript of the pmirGLO Vector (E1330, Promega) by external services (GenScript). The Renilla luciferase transcript will not include any SARS-COV-2 elements, being the endogenous control.
(b) For the N-protein expression vector, the SARS-COV-2 N-protein (1) mRNA complete sequences and (2) 3′UTR paring sequences mutated mRNA will be synthetized and cloned in the phMGFP vector (E6421, Promega) by external services (GenScript). These designs will allow the discrimination between 3′UTR and 5′UTR-CDS regulation capabilities of the anti-SARS-CoV-2 designed miRNA mimics. On the other hand, the plasmid pUNO1-SARS2-S-d19 (puno1-cov2-sd19, In VivoGen) will be used as the SARS-COV-2 S protein expression vector.

### Transfection

Lipofectamine3000 (L3000015, ThermoFisher) may have been used for transient expression of the plasmid constructs on the HELA cell-lines following manufacturer instructions.

### Luciferase Assay

Luciferase assays were performed using the Dual-Glo® Luciferase Assay System (E2940, Promega) following manufacturer instructions.

### Eukaryotic Expression Assay

### RT-qPCR

Total RNA may be extracted from cell-lines by using the PureLink RNA extraction minikit (12183020, ThermoFisher) per manufacturer instructions and, RT-qPCR will be carried out by using the SuperScript™ III One-Step RT-PCR System kit (12574026, ThermoFisher) per manufacturer instructions in a QuantStudio 3 Real-Time PCR System (ThermoFisher). The expression levels of three NormFinder validated (Andersen C. L., Ledet-Jensen J., Ørntoft T.: Normalization of real-time quantitative RT-PCR data: a model based variance estimation approach to identify genes suited for normalization—applied to bladder- and colon-cancer data-sets. Cancer Research. 2004 (64): 5245-5250) reference genes were quantified (GAPDH, PPIA and RPS13) along either the levels of the S or N mRNA, depending of the plasmid the cells were transfected with. For the diferential expression analysis, the S and N expression levels were normalized using the REST formula as described in Hellemans, J., Mortier, G., De Paepe, A., Speleman, F. & Vandesompele, J. qBase relative quantification framework and software for management and automated analysis of real-time quantitative PCR data. Genome Biol. 8, (2008).

### Western Blot

Total protein may be extracted from cell-lines by using RIPA Buffer (R0278, Sigma-Aldrich) and Protease Inhibitor Cocktail (P8340, Sigma-Aldrinch) following the recommended protocol. Total protein quantification will be done by using Pierce™ BCA Protein Assay Kit (23227, Thermo Fisher) per manufacturer instructions.

### Evaluation of Viral Titer in SARS-COV-2 In Vitro Model Using Live Virus

SARS-COV-2 (accession: NC_045512.2) stock may be cultured following conditions previously described⁴⁴. Virus stocks may be titrated on Vero E6 cells (ATCC® CRL-1586™), in a biosafety level-4 (BSL-4) facility. Virus titration assay may be done following the protocol previously described⁴⁵. Virus titter may be calculated using the formula: PFU/mL=number of plates counted/(inoculation volume (50 uL in this case)*sample dilution)

This work may be performed through external services or collaborations. RT-qPCR will be used to confirm viral RNA loads and the specific identity of SARS-COV-2. Standard and approved methodologies for amplification of viral RNA may be used.

### Dose-Range Finding Safety Study in an Outbred Murine Model

CD-1 mice (CD-1® IGS Mice, Charles River) may be used as experimental subjects. Mice may be grouped according to their weight. The experimental subjects may be treated intravenously every other day with a determined dosing amount in a final injection volume of 200 uL for one month. Once the treatment regimen is completed, blood parameters including but not limited to reference factors for hepatic and kidney functions, may be evaluated as the safety reference. Pathology analysis for various body tissues may be performed by a certified veterinary pathologist.

### Efficacy Study in a Transgenic Murine or Other Animal Model

A novel transgenic mouse model is being developed for studying SARS-COV-2 but is not yet widely available. If this model becomes available, we may propose its use. In this case, specific pathogen-free, 6-11-month-old, female wild type (WT) C57BL/6J (000664) and, transgenic (TG) K18-hACE2 (034860) mice may be obtained from The Jackson Laboratory. Two experiments may be run:
(a) Nanoparticle formulation delivery: SARS-COV-2 free WT and TG mice will be dosed intravenously in a q.o.d regimen (M, W, F) with the nanoparticle formulation containing an Anti-SARS-COV-2 miRNA mimic. After completion of treatment, mice will be sacrificed, and Anti-SARS-COV-2 miRNA mimic will be quantified on the respiratory system by RT-qPCR.
(b) Efficacy study: TG mice will be inoculated intranasally with SARS-COV-2 at a dosage of 10⁵ TCID₅₀. After confirmed infection, mice will be separated in two groups: (1) Intravenously dosing of the nanoparticle formulation containing an Anti-SARS-COV-2 miRNA mimic in a q.o.d regimen (M, W, F) and, (2) Intravenously dosing of the nanoparticle formulation containing an scramble miRNA mimic in a q.o.d regimen (M, W, F). Animals will be continuously observed daily to record body weights, clinical symptoms, responsiveness to external stimuli and death.

If this model is not available or an alternative animal model is deemed more appropriate, an adequate alternative research methodology may be proposed.

### Human Blood Immune Response Ex Vivo Assay

To determine whether the formulated nanoparticle induces a human cytokine response, it may be incubated for 24 hours in whole blood collected from healthy volunteers. Anticoagulant may be added to the whole blood to prevent coagulation and the formulated nanoparticle may be tested at three different concentrations. After the incubation, the plasma may be separated by centrifugation and IL-6, INFα, and TNFα will be analyzed by ELISA (KHC0011, BMS223HS, KHC0121, BMS213HS, ThermoFisher).

Thus, a series of RNAi-inducing siRNA/miRNA molecules may be designed to specifically target the SARS-COV-2 viral RNA at different locations. The sequences shown below in Table 3 have been synthesized, and have obtained results for the first candidate, labeled as “D”. The scrambled, non-effective sequence “NC” is used as a “negative control.”

TABLE 9

| RNAi Identifier | Sequence |
|---|---|
| Anti-SC2-A | UCACCAUUACUAGGUUUG |
| Anti-SC2-B | UGUUGAGUCAGAGCUAUG |
| Anti-SC2-C | UCAUUAGUAGGGUUGA |
| Anti-SC2-D | UUCACUGUACACUCGA |
| Anti-SC2-E | UUUACACAUUAGGGCU |

Sequence “D” was tested for efficacy against a luciferase reporter construct, designed to include the 3′UTR (untranslated region) of the SARS-COV-2 RNA, as shown below:

SARS-COV-2 3′UTR
CAATCTTTAATCAGTGTGTAACATTAGGGAGGACTTGAAAGAGCCACCAC
ATTTTCACCGAGGCCACGCGGAGTACGATCGAGTGTACAGTGAACAATGC
TAGGGAGAGCTGCCTATATGGAAGAGCCCTAATGTGTAAAATTAATTTTA
GTAGTGCTATCCCCATGTGATTTTAATAGCTTCTTAGGAGAATGACAAAA
AAAAAAAAAAAAAAAAAAAAAAAAAAAAA

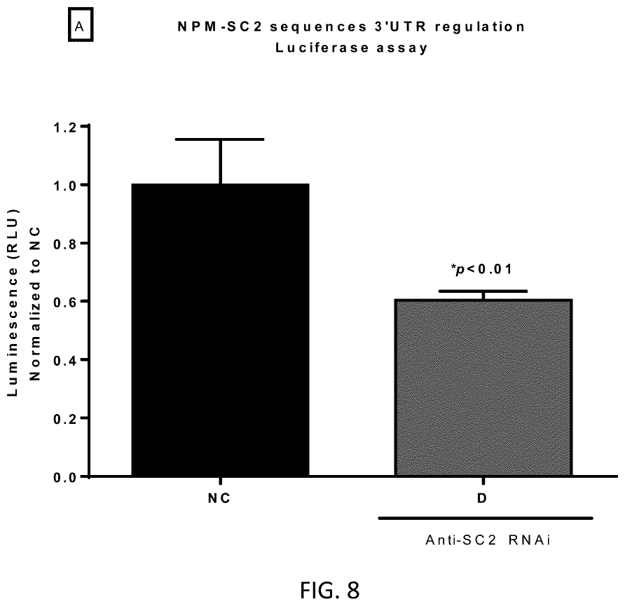

293T cells were transfected with both the luciferase reporter construct and the antiviral sequence “D,” as described above. Following forty-eight hours, the luciferase expression of the reporter construct was examined using a plate reader. Observed was a significant reduction in luciferase activity following transfection of sequence “D,” demonstrating the regulatory potential and efficacy of the designed sequence. These results are shown in FIG. 8.

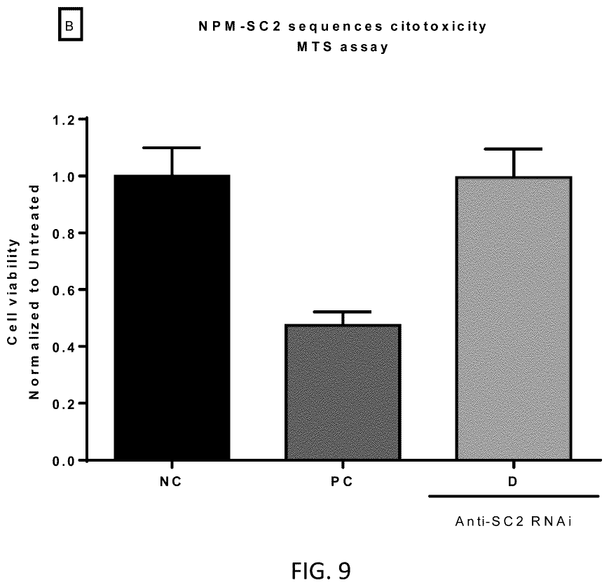

Potential cytotoxic effects of sequence “D” in fibroblasts were also tested. The cells were transfected with either a positive cytotoxic control, the NC, or sequence “D.” No cytopathic effects were observed through MTS assay at 48 hours post transfection, indicating the relative safety of the sequence. This is shown in FIG. 9.

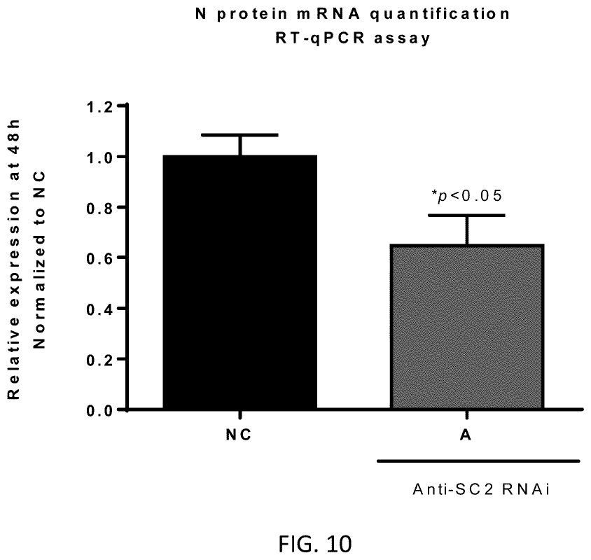

HeLa cells were transfected with both SARS-COV-2 N protein expression construct and the antiviral sequence “A,” as described above. Following forty-eight hours, the expression of the protein N mRNA was examined using qPCR. Observed was a significant reduction in protein N mRNA following transfection of sequence “A” demonstrating the regulatory potential and efficacy of the designed sequence. These results are shown in FIG. 10.

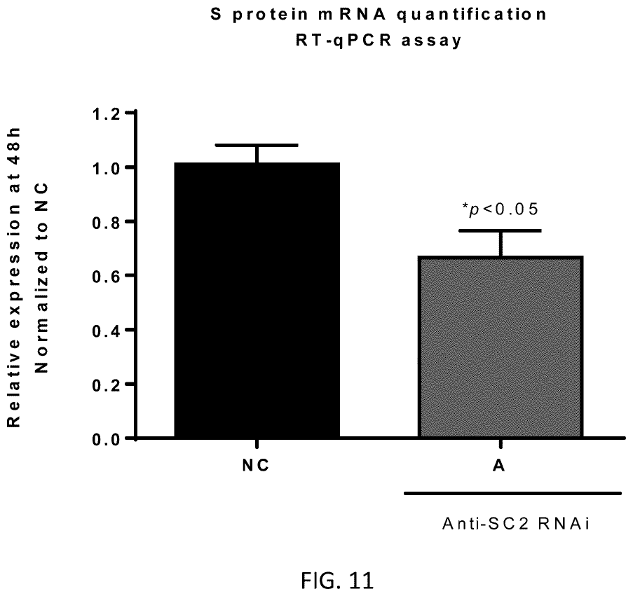

HeLa cells were transfected with both SARS-COV-2 S protein expression construct and the antiviral sequence “A,” as described above. Following forty-eight hours, the expression of the protein N mRNA was examined using qPCR. Observed was a significant reduction in protein N mRNA following transfection of sequence “S” demonstrating the regulatory potential and efficacy of the designed sequence. These results are shown in FIG. 11.

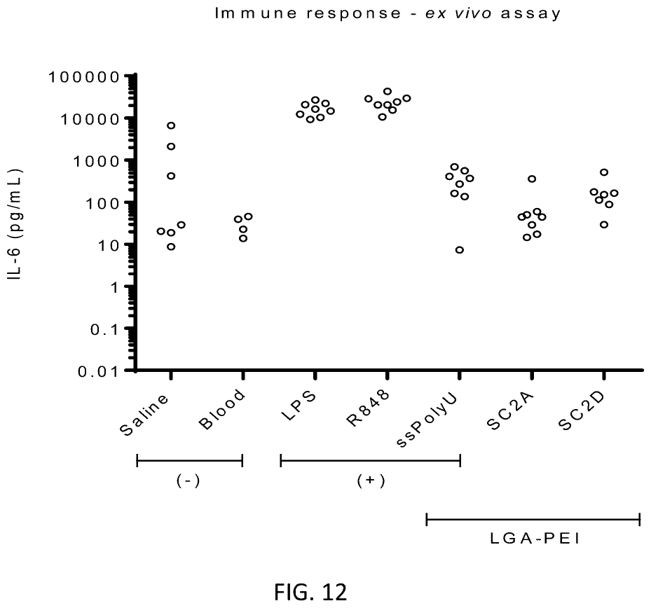

Human immune response was tested for the antiviral sequences A and D as describe above. Following forty-eight hours of incubation/exposure, concentration of IL-6, TNFα and INFa were examined. No significant immune response was observed after exposure to sequence A or D when comparing with negative controls (−). These results are shown in FIG. 12.

While this disclosure has been described in conjunction with the embodiments outlined above, many alternatives, modifications and variations will be apparent to those skilled in the art upon reading the foregoing disclosure. Accordingly, the embodiments of the disclosure, as set forth above, are intended to be illustrative, not limiting. Various changes may be made without departing from the spirit and scope of the disclosure.

What is claimed is:

1. A method, comprising:
selecting an RNA or a set of RNAs;
defining a minimum and a maximum target RNAi transcript hybridization length of the RNA or set of RNAs; and
executing a computer algorithm,
wherein the computer algorithm determines either a most abundant nucleotide sequences in the transcript or set of transcripts with a length that matches the minimum target RNAi transcript hybridization length.

2. The method of claim 1, further including:
selecting an RNAi guide sequence based on an index that represents a probability of an RNAi having silencing capabilities against the RNA or set of RNAs.

3. The method of claim 1, wherein only nucleotide sequences with a GC content higher than 35% are considered.

4. The method of claim 1, wherein, for each of the nucleotide sequences generated, a potential reverse complementary guide strand sequence of the RNAi is generated.

5. The method of claim 4, wherein the potential reverse complementary guide strand sequence of the RNAi is generated by producing an average nucleotide sequence with the maximum target RNAi transcript hybridization length from the nucleotide sequences of a maximum length at sites where a given small nucleotide sequence is present in the transcript or set of transcripts.

6. The method of claim 4, wherein each of the potential reverse complementary guide strand sequence of the RNAi is qualified based on certain characteristics, the certain characteristics including:
a hit feasibility index; or
a custom index,
the custom index representing a probability of a given potential hybridization site to have biological significance, or a probability of an RNAi having silencing capabilities against the RNA.

7. A system, the system comprising:
a computer algorithm,
wherein the computer algorithm determines either a most abundant nucleotide sequences in a transcript or set of transcripts with a length that matches a minimum target RNAi transcript hybridization length, and
wherein the minimum target RNAi transcript hybridization length and a maximum target RNAi transcript hybridization length of the RNA or set of RNAs are defined from a selected RNA or a set of RNAs.

8. The system of claim 7, wherein the computer algorithm further selects an RNAi guide sequence based on an index that represents a probability of an RNAi having silencing capabilities against the RNA or set of RNAs.

9. The system of claim 7, wherein only nucleotide sequences with a GC content higher than 35% are considered.

10. The system of claim 7, wherein, for each of the nucleotide sequences generated, a potential reverse complementary guide strand sequence of the RNAi is generated.

11. The system of claim 10, wherein the potential reverse complementary guide strand sequence of the RNAi is generated by producing an average nucleotide sequence with the maximum target RNAi transcript hybridization length from the nucleotide sequences of a maximum length at sites where a given small nucleotide sequence is present in the transcript or set of transcripts.

12. The system of claim 10, wherein each of the potential reverse complementary guide strand sequence of the RNAi is qualified based on certain characteristics, the certain characteristics including:
a hit feasibility index; or
a custom index,
the custom index representing a probability of a given potential hybridization site to have biological significance, or a probability of an RNAi having silencing capabilities against the RNA.

13. A computer-readable storage medium having data stored therein representing software executable by a computer, the software having instructions to:
determine either a most abundant nucleotide sequences in a transcript or set of transcripts with a length that matches a minimum target RNAi transcript hybridization length,
wherein the minimum target RNAi transcript hybridization length and a maximum target RNAi transcript hybridization length of the RNA or set of RNAs are defined from a selected RNA or a set of RNAs.
